(19) United States
(12) Patent Application Publication
(10) Pub. No.: US 2011/0040180 A1
(43) Pub. Date: Feb. 17, 2011

(54) Method and device for determining the position of a tangential plane on three extreme points of a body

(75) Inventors: François Leitner, Uriage (FR); Jean-Baptiste Pinzuti, Voreppe (FR); Benoît Mollard, Echirolles (FR); Thomas Lemaire, Fontaine (FR)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/804,202

(22) Filed: Jul. 14, 2010

(30) Foreign Application Priority Data
Aug. 12, 2009 (DE) 10 2009 037 208.3

(51) Int. Cl. A61B 8/00

(52) U.S. Cl. 600/437

(57) ABSTRACT

In order to improve the computation of the position of the tangential plane in a method for determining the position of a tangential plane which rests tangentially on three non-collinear extreme points of structures of a body, in which one determines the position of the three extreme points approximately by using three starting points disposed in the locality of the structures and computes the tangential plane as being a plane passing through these three starting points, it is proposed that one investigates the structure of the body in the vicinity of the three extreme points using a navigated ultrasonic head which emits ultrasonic radiation in an investigation plane and displays the ultrasonic image of each investigated structure on a display screen, in that one overlays a line of intersection of the investigation plane and the tangential plane on the ultrasonic image in the display screen, in that, for each structure, one selects as an improved extreme point that point of the structure which is located in the investigation plane at the greatest or the least spacing from the line of intersection, and in that one computes an improved tangential plane as being a plane which passes through these three improved extreme points. A device for carrying out this method is disclosed in the patent.

This application claims the benefit of German Patent Application No. 10 2009 037 208.3 filed on Aug. 12, 2009.

The present disclosure relates to the subject matter disclosed in German patent application No. 10 2009 037 208.3 of Aug. 12, 2009, which is incorporated herein by reference in its entirety and for all purposes.

### BACKGROUND OF THE INVENTION

The invention relates to a method for determining the position of a tangential plane which rests tangentially on three non-collinear extreme points of structures of a body, wherein one determines the position of the three extreme points approximately by means of three starting points disposed in the locality of the structures and computes the tangential plane as being a plane passing through these three starting points. Moreover, the invention relates to a device for carrying out a method of this type.

When determining the position of certain planes in the body, these are frequently selected in such a way that they rest tangentially on extreme points of a structure. One example of such a plane is the pelvic inlet plane which rests tangentially on the two upper, front illiac crests and on the symphysis pubis. Exact locationing of this plane is only possible if the extreme points of the structure on which the plane rests tangentially can be determined precisely.

In practice, one tries to get close to them in that one either feels for the salient structures of the body with the help of a navigated stylus-type instrument for example, or else one scans the structures with an ultrasonic head and displays the thus obtained ultrasonic image on a display screen, whereupon the operator selects the position of the extreme point on the body structure depicted on the display screen. In both cases however, this determination of the extreme points can only be effected approximately and the result is usually subject to a greater or lesser degree of error, for example, when probing for the extreme points, due to the differing layers of soft tissue and accumulations of fat in the vicinity of the probed structures, or, in the case of the ultrasonic investigation, due to an inaccurate determination of the extreme points on the display screen.

The object of the invention is to provide a method wherein the process of determining the position of these extreme points and thus of the tangential plane passing through the extreme points can be improved.

### SUMMARY OF THE INVENTION

In accordance with the invention, this object is achieved in the case of a method of the type mentioned hereinabove in that one investigates the structure of the body in the vicinity of the three extreme points using a navigated ultrasonic head emitting ultrasonic radiation in an investigation plane and displays the ultrasonic image of each investigated structure on a display screen, in that one overlays a line of intersection of the investigation plane and the tangential plane on the ultrasonic image in the display screen, in that one selects as an improved extreme point for each structure that point of the structure lying in the investigation plane which is at the greatest or least spacing from the line of intersection, and in that one computes a plane passing through these three improved extreme points as being the improved tangential plane.

Thus, the line of intersection with the tangential plane which has been computed on the basis of the as yet imprecisely determined starting points is superimposed on the ultrasonic image of the ultrasonic head which corresponds to the investigation plane, and this intersection line is used as a basis for finding the point of the imaged structure which is at the greatest or smallest distance from this intersection line. Whether the point at the greatest distance or the one at the least distance from the intersection line is selected depends on the position of the intersection line relative to the bony structure. If the concave inner side of the bony structure faces the intersection line, then the point of this section of the bony structure at the greatest distance is selected, whereas, if the convex outer side of the bony structure faces the intersection line, then that point which is at the least distance from the intersection line is selected. In this way, the point of the curved bony structure which is located at an extreme distance from the intersection line can be determined in each case, i.e. the point which exhibits an extreme value of the curved shape of the bony structure relative to the intersection line.

An improved extreme point is determined in the same way for each of the structures, and these new improved extreme points are used in order to compute a plane which passes through them all, this then being used as the tangential plane. In following this line of procedure, it has been discovered that even if the choice of starting points is inaccurate, the improved extreme points correspond very much closer to the actual extreme points than the originally utilised starting points. In consequence, the position of the tangential plane determined in this way is substantially closer to the actual extreme points of the body structures compared with the tangential plane that was determined on the basis of the starting points.

This method can be repeated using starting points that have not been determined as starting points utilising other methods, but rather, the improved extreme points obtained from the method described above. With the aid of this iterative process, one obtains in the next step a further improvement in the position of the extreme points vis a vis the starting points which, for their part, were already improved extreme points coming from the preceding computational process.

It has been demonstrated that a convergence arises when these process steps are repeated a number of times, i.e. the tangential plane that has been determined in this way corresponds to the optimal tangential plane passing through the actual extreme points after just a few passes.

One can determine the original starting points at the beginning of the method in known manner with the help of a navigated probe for example or else with the help of the navigated ultrasonic head and the display screen, in this case the starting point is simply selected visually on the display screen.

The invention also relates to a device for determining the position of a tangential plane which rests tangentially on three non-collinear extreme points of structures of a body comprising a navigated ultrasonic head which emits ultrasonic radiation in an investigation plane, a display screen for displaying the ultrasonic image of a structure of the body that has been investigated with the ultrasonic head and also a data processing system.

In order to improve the process of determining the tangential plane here, it is proposed in accordance with the invention that the data processing system be programmed in such a way that, from three starting points which are disposed in the locality of the structures and correspond approximately to extreme points, it computes a tangential plane passing through the three starting points, in that it overlays a line of intersection resulting from the intersection of the investigation plane and the tangential plane on the representation of the ultrasonic image of each structure on the display screen, and in that it computes an improved tangential plane passing through the improved extreme points which correspond to those points of each structure which are located in the investigation plane at the greatest or the least distance from the intersection line.

In accordance with a preferred embodiment, provision may be made for the data processing system to be programmed in such a way that it repeats the process steps of Claim 4 using the improved extreme points as starting points.

It is advantageous if a navigated probe is provided for determining the starting points at the beginning of the method.

The following description of preferred embodiments of the invention will serve to provide a more detailed explanation taken in conjunction with the drawing.

### BRIEF DESCRIPTION OF THE DRAWINGS

FIG. 1: is a perspective view of a patient lying on a couch including an ultrasonic head for investigating the salient structures of the pelvis;

FIG. 2: is a schematic view of an ultrasonic head, of three structures of the body and of a plane which passes through starting points in the locality of the body structures which do not correspond to the actual extreme points;

FIG. 3: is a display screen with an ultrasonic image of an arc-shaped body structure and an intersection line of the ultrasonic investigation plane and the tangential plane superimposed on the display screen and

FIG. 4: is a view similar to FIG. 3 wherein the body structure bears a marking in the form of a circle.

### DETAILED DESCRIPTION OF THE INVENTION

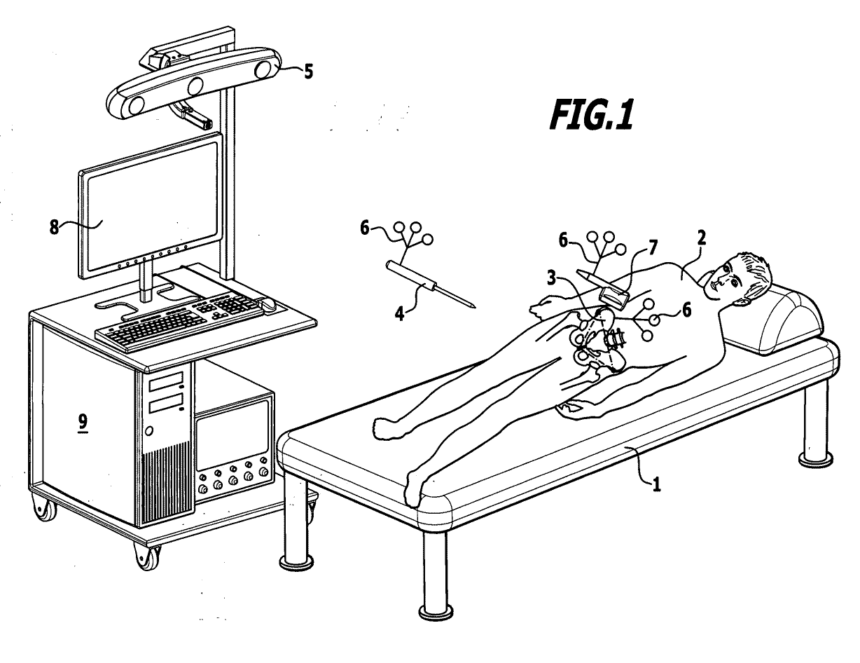

A patient 2 whose pelvic bone 3 is being investigated with the help of an ultrasonic head 4 is illustrated lying on a couch 1 in FIG. 1. The invention will be explained using the example of a process for determining the pelvic inlet plane, a plane which is determined by the spina iliaca superior (front upper illiac crest) of the pelvic bone and by the symphysis pubis (pubic bone). This relates to the salient structures of the pelvic bone which can be probed to an approximate extent through the skin, for example, with the help of a navigated probe 4 the point of which can be placed on the structure and can thus determine the position of the structure in space.

For the purposes of navigating this probe 4 and also the pelvic bone 3, there is provided a known navigation system 5 which can determine the position of marking elements 6 in space, these marking elements 6 being connected firmly to the probe 4 and the pelvic bone 3 so that the position of the probe 4 and the pelvic bone 3 in space can also be ascertained thereby. Here, the expression position is to be understood as including both the location and the orientation in space.

With the help of the navigated probe 4, the said three extreme points on the pelvic bone can be determined, although only to an approximate extent, since these extreme points cannot be determined with the requisite accuracy possibly due to the probe not being locationed properly or else as a result of difficulties which arise as a result of the distribution of the soft tissue and the layers of fat in the vicinity of the pelvis. Thus, one only obtains approximate values for the extreme points in this way, these approximate values being referred to hereinafter as starting points.

In order to determine the actual extreme points or at least an improved version thereof, an ultrasonic head 7 is used in the context of the method being described here, said head emitting ultrasonic radiation in a plane and then receiving the reflected ultrasonic radiation so that the distances of the reflecting structures can be determined from the elapsed time differences. These reflecting structures are, for example, the illiac crests of the pelvic bone so that the course thereof relative to the ultrasonic head is capable of being determined in this way.

The ultrasonic head 7 likewise carries a marking element 6 so that its position in space can also be determined by the navigation system 5.

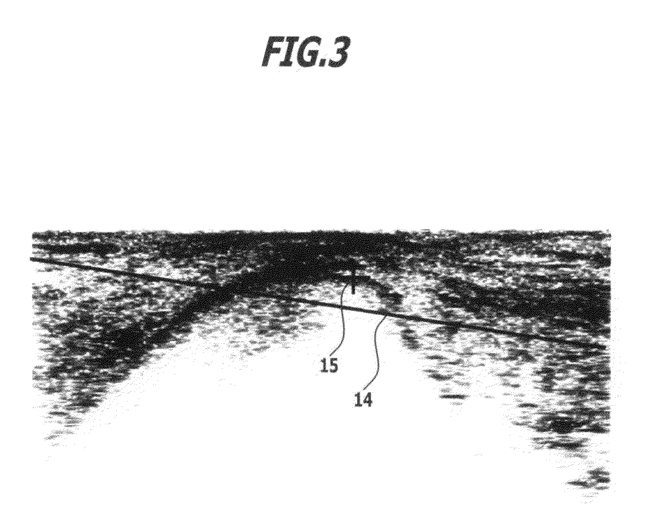

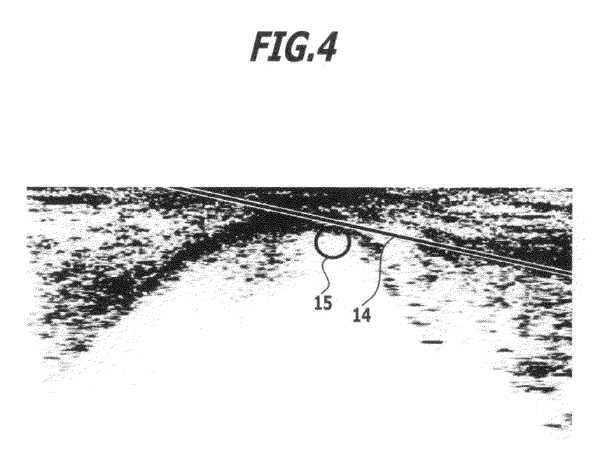

The ultrasonic image produced by the ultrasonic head is displayed on a display screen 8, and on this for example, one can perceive the arc-shaped form of an illiac crest such as is illustrated in FIGS. 3 and 4.

One can immediately appreciate from this structure that it is extremely difficult to determine an accurate extreme point on the basis of this representation. However, one could also use this ultrasonic image in order to determine an approximate extreme point, i.e. a starting point. Thereby, the operator takes one point on the curve of the illustrated structure and assumes that this is the extreme point being sought. This too is merely an estimation, but this determination can, for example, replace the one determined by the navigated probe 4. Since the ultrasonic head is being navigated, the position of the starting point determined in this manner can also be determined in this way.

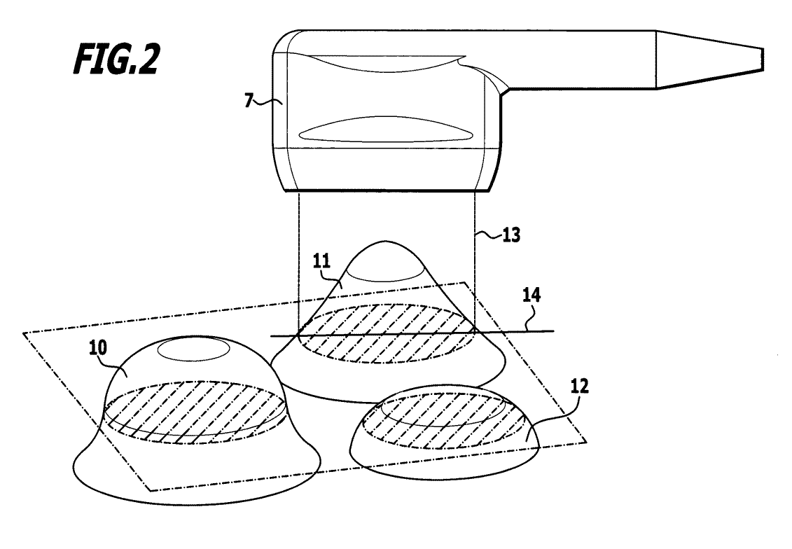

From the starting points that have been obtained in this way, these merely being approximate values for the extreme points being sought, a data processing system 9 can determine a plane which passes through the three starting points. This plane is illustrated in FIG. 2 for example, one can perceive that this plane which has been determined on the basis of approximate values of the extreme points does not actually rest tangentially on the extremities of the structures 10, 11, 12, but rather, intersects them. The reason for this is that the starting points do not coincide with the extreme points, but are merely located in the proximity thereof.

From this plane passing through the starting points on the one hand and the investigation plane 13 of the ultrasonic head 7 on the other, the data processing device 9 computes an intersection line 14, and this is superimposed on the image display screen 8 in such a way that it is overlaid on the ultrasonic image produced by the ultrasonic head 7. In the illustration of FIG. 3, it is clear that this intersection line 14 intersects the structure of the body being investigated, although it is not generally tangential thereto since the starting points do not correspond to the actual extreme points, but are merely an approximation thereto.

In the next step, an extreme point is selected in each structure which has been investigated and displayed on the display screen in this way, this extreme point being the point of the structure, thus for example, the arc-shaped image of the illiac crest, which is located at the greatest spacing from the intersection line 14. This extreme point 15 is marked in FIG. 3 with a cross so that it is accurately defined.

In the case of the exemplary embodiment depicted in FIG. 3, the arc-shaped bony structure which is shown in the ultrasonic image is arranged relative to the intersection line 14 in such a way that the concave inner part of an arc-shaped section of this bony structure is associated with the intersection line 14, i.e. the intersection line is located under an extreme point 15 and in this case intersects the arc-shaped bony structure in two places. In this case, the point selected as the extreme point is the point located at the greatest spacing from the intersection line.

It would also be possible for the intersection line 14 to be arranged above the point 15 so that the convex outer part of the arc-shaped structure faces the intersection line and the latter does not intersect the arc-shaped structure. In this case, the point at the greatest spacing from the intersection line would not be selected as the extreme point, but rather, it would be the point with the smallest spacing. If the intersection line 14 does not run below the extreme point 15 as illustrated in FIG. 3, but parallel to the intersection line 14 although above the extreme point 15, then the same extreme point of the bony structure would be selected in this way.

In the exemplary embodiment depicted in FIG. 4, a region of the structure being investigated is marked with a geometrical figure, in this case by means of a circle, this circle can, for example, be placed tangentially on the structure. One thereby has a greater degree of certainty in regard to the locationing of this marking as compared with the locationing of a cross, the latter must be placed at one point, whereas the circle can be placed tangentially relative to a structure so that the position of the circle does not determine just one point of the structure, but rather, a larger number of points of the structure in the region of application.

In both cases however, the position of the extreme point which is located in the plane of investigation 13 and is at the greatest spacing from the intersection line 14 can be determined from the position of the cross or the circle.

Three extreme points which are a substantially closer approximation to the actual extreme points of the structures than the starting points are determined in this way. The extreme points determined in this way are then used for the computation of a new tangential plane, the latter will still not normally pass exactly through the extreme values of the structures, i.e. it is still not resting exactly tangentially on the structures, but the course of this tangential plane will be substantially closer to the actual extreme points than was the case for the initial plane passing through the starting points.

The process described above can be repeated by using as the new starting points for this process those extreme points which were determined in the last implementation thereof. As a result of repeating the process in this way, a continuous improvement in the position of the specific extreme points and thus in the position of the tangential plane passing through these extreme points is obtained.

It has been shown that this method converges so that after just a few steps one will obtain a plane which is really tangential to the structures under investigation and the position of which will remain practically unchanged by further implementations of the method.

Thus, by using this method even with a relatively inaccurate determination of the starting points, a plane which does not pass through the inaccurately determined starting points but one which is substantially exactly tangential to the structures of the body can be computed in a very simple way, thus for example, an accurate pelvic inlet plane.

Due to the superimlocation of the intersection line, the operator is shown the direction away from the intersection line in which to search for the extremities of the structure, i.e. in a direction perpendicular to the extent of the intersection line, since it is the maximum spacing from the intersection line which is determined. This direction will change with each pass, since the position of the tangential plane passing through the three extreme points approximates more closely to the position of the final tangential plane after each pass.

1. A method for determining the position of a tangential plane which rests tangentially on three non-collinear extreme points of structures of a body, wherein one determines the position of the three extreme points approximately by using three starting points disposed in the locality of the structures and computes the tangential plane as being a plane passing through these three starting points, wherein one investigates the structure of the body in the vicinity of the three extreme points with a navigated ultrasonic head which emits ultrasonic radiation in an investigation plane and displays the ultrasonic image of each investigated structure on a display screen, one overlays a line of intersection of the investigation plane and the tangential plane on the ultrasonic image in the display screen, in that, for each structure, one selects as an improved extreme point that point of the structure which is located in the investigation plane at the greatest or the least spacing from the line of intersection, and one computes an improved tangential plane as being a plane passing through these three improved extreme points.

2. A method in accordance with claim 1, wherein one selects the improved extreme points as starting points and repeats the method of claim 1 with these new starting points.

3. A method in accordance with claim 1, wherein one determines the starting points at the beginning of the method with the help of a navigated probe or with the help of a navigated ultrasonic head.

4. A method in accordance with claim 2, wherein one determines the starting points at the beginning of the method with the help of a navigated probe or with the help of a navigated ultrasonic head.

5. A device for determining the position of a tangential plane which rests tangentially on three non-collinear extreme points of structures of a body, comprising a navigated ultrasonic head which emits ultrasonic radiation in an investigation plane, a display screen for displaying the ultrasonic image of a structure of the body that has been investigated with the ultrasonic head and also a data processing system, wherein the data processing system is programmed in such a manner that, from three starting points which are disposed in the locality of the structures and correspond approximately to the extreme points, it computes a tangential plane passing through these three starting points, it overlays a line of intersection resulting from the intersection of the investigation plane and the tangential plane on the representation of the ultrasonic image of each structure on the display screen, and it computes an improved tangential plane passing through the improved extreme points which correspond to those points of each structure which are located in the investigation plane at the greatest or the least spacing from the line of intersection.

6. A device in accordance with claim 5, wherein the data processing system is programmed in such a way that it repeats the process steps of claim 5 using the improved extreme points as starting points.

7. A device in accordance with claim 5, wherein a navigated probe is provided for determining the starting points at the beginning of the method.

8. A device in accordance with claim 6, wherein a navigated probe is provided for determining the starting points at the beginning of the method.
